(19) United States
(12) Patent Application Publication
(10) Pub. No.: US 2004/0111105 A1
(43) Pub. Date: Jun. 10, 2004

(54) Cleaning device for body piercings

(75) Inventors: Rev. Suzanne M. Schmieding, Boothwyn, PA (US); Dyer Harris, Wilmington, DE (US)

Correspondence Address: KRAMER & AMADO, P.C., 2001 JEFFERSON DAVIS HWY, SUITE 1101, ARLINGTON, VA, 22202, US

(73) Assignee: Rev. Suzanne M. Schmieding, Boothwyn, PA

(21) Appl. No.: 10/310,946

(22) Filed: Dec. 6, 2002

(51) Int. Cl. A61F 9/00

(52) U.S. Cl. 606/162

(57) ABSTRACT

The present invention relates to an effective and efficient cleaning device for body piercings anywhere on the body. The cleaning device involves a non-fibrous core that is tightly wrapped with a fibrous material. The cleaning device has a leading end and a following end, where the leading end has a tip that facilitates insertion into the area to be cleaned.

### BACKGROUND OF THE INVENTION

1. Field of the Invention

This invention relates generally to a cleaning device. More particularly, the invention relates to a cleaning device for cleaning body piercings.

2. Description of Related Art

As body piercing continues to gain in popularity, it has gone beyond the traditional area of ear piercing. Piercing of the ears, nose, eyebrows, lip, tongue and other body parts is now a common practice all over the world. However along with the increase in body piercings there is a corresponding increase in infections, rashes, inflammation, allergic reactions, and other painful ailments, caused by a failure to maintain proper hygiene of pierced areas.

The lack of proper hygiene may result from a failure of the health care or body piercing professional to use proper procedures when piercing the body part. The absence of proper hygiene may also result from the consumer lacking the necessary tools to properly clean and maintain their body piercing. Over time, a number of irritants, such as dirt, residue, creams, lotions, sweat, soap, shampoo, dead skin cells, body fluids and blood, as well a particles from costume jewelry and the like, may build up in the body piercing cavity. These irritants can lead to inflammation or provide a base for future infection. In order to provide for the proper health of the body piercing, it is essential that the consumer remove these irritants in a gentle and effective manner on a daily basis.

Therefore, there is a need to provide a simple yet effective device for cleaning and maintaining proper hygiene for all body piercings, not just ear piercings. The device must be easy to use and designed for maximum comfort so that consumers of all ages are encouraged to use the device as part of a daily routine to maximize the device's effectiveness. Additionally, the device should be relatively inexpensive to produce so that the device is affordable both to the home consumer and health care or body piercing professionals.

Information relevant to attempts to address some of these problems can be found in U.S. Pat. Nos. 4,041,946, 4,353,3720, 4,497,402, 4,798,216, 6,146,398 and 6,358,221. However, each one of these references suffers from one or more of the following disadvantages: 1. Many of the references are particularly designed to clean or medicate pierced earlobes and are not suitable to be used on all body piercings generally. 2. Other references describe devices that are left in the body piercing for a lengthy period of time, or are designed to be moved back and forth in the body piercing. 3. Some references are not designed with the consumer's comfort in mind or are increasingly complex and may be difficult for the consumer to use properly. 4. A number of references disclose structures that are impractical or expensive to produce.

The foregoing objects and advantages of the invention are illustrative of those that can be achieved by the present invention and are not intended to be exhaustive or limiting of the possible advantages which can be realized. Thus, these and other objects and advantages of the invention will be apparent from the description herein or can be learned from practicing the invention, both as embodied herein or as modified in view of any variation which may be apparent to those skilled in the art. Accordingly, the present invention resides in the novel methods, arrangements, combinations and improvements herein shown and described.

### SUMMARY OF THE INVENTION

In light of the present need for an effective and efficient cleaning device for human body piercings anywhere on the human body, a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

The present invention relates to an effective and efficient cleaning device for body piercings anywhere on the human body. The cleaning device involves a non-fibrous core that is tightly wrapped with a fibrous material. The cleaning device has a leading end and a following end, where the leading end has a rounded tip for greater comfort and ease of insertion into the area to be cleaned. The non-fibrous core is rigidly flexible in that it is rigid enough to be pushed into and through body piercings, while at the same time, flexible enough to easily move through curved areas. All of the materials used to make the product are non-toxic and may be hypoallergenic so that there is no irritation of the areas that the cleaning device is inserted into.

The cleaning device is packaged and sterilized. It may also be combined with a medicant before or after packaging.

The consumer uses the cleaning device by removing it from the sterile packaging, then inserting the leading end into the area to be cleaned. The leading end is pushed through the body piercing. As the leading end is guided through the body piercing, the user may push the device in a slightly rotating motion, or a very slow gentle spinning motion. The user then pulls the cleaning device by the leading end through the area to be cleaned, in one direction only, until the following end exits the area to be cleaned. The same rotating or spinning motion as stated above may be used while pulling the device through the body piercing. This simple yet effective device may be used at home, at professional piercing establishments, by healthcare professionals, or the jewelry, cosmetology, and healthcare industries generally.

### DESCRIPTION OF THE DRAWINGS

In order to better understand the present invention, reference is made to the accompanying drawings, wherein:

FIG. 1 shows a perspective view of the cleaning device.

FIG. 2 shows a close-up view of the formation of the rounded tip on the leading end of the device.

FIG. 3 shows a flow chart of the method for making the device.

### DETAILED DESCRIPTION OF THE PREFERRED EMBODIMENTS OF THE INVENTION

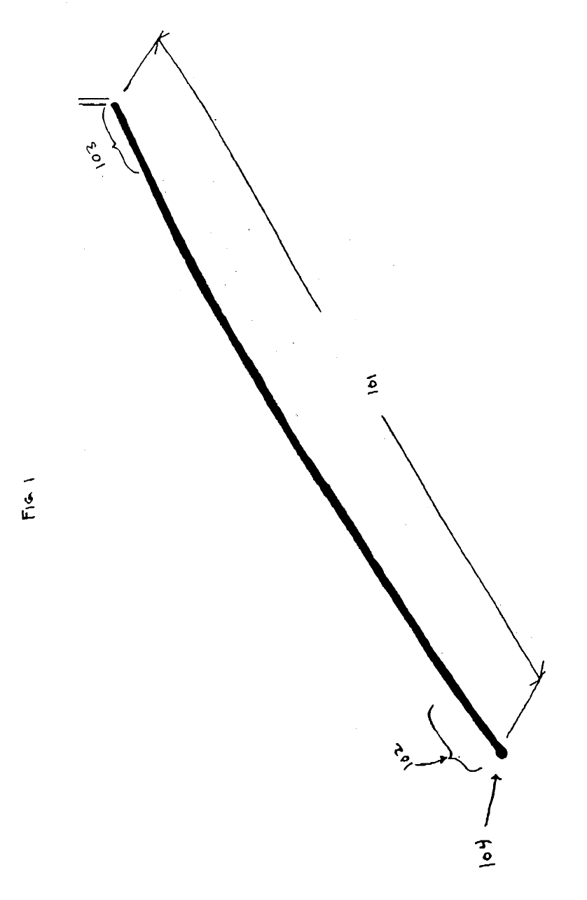

Referring now to the drawings, in which like numerals refer to like components or steps, there are disclosed broad aspects of the preferred embodiments of the present invention. FIG. 1 shows a perspective view of the cleaning device. The cleaning device has a non-fibrous core wrapped with a fibrous material 101, that includes a leading end 102 and a following end 103. The leading end 102 has a rounded tip 104 to be inserted into the area to be cleaned.

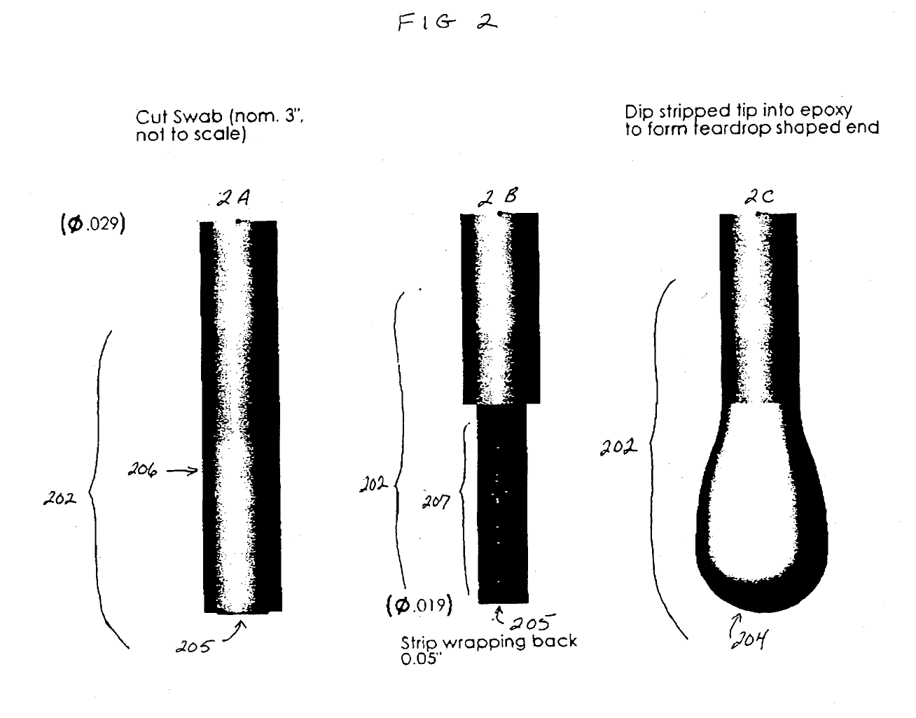

FIG. 2 shows a close up view of the rounded tip 204 and the steps for making the tip. FIG. 2A shows the non-fibrous core 205, with the fibrous material 206 wrapped around it at the leading end 202 of the cleaning device. FIG. 2B shows stripped area 207 where the wrapping has been removed from the non-fibrous core 205 at the leading end 202. The rounded tip 204 is formed at the location of the stripped area 207 on the leading end 202.

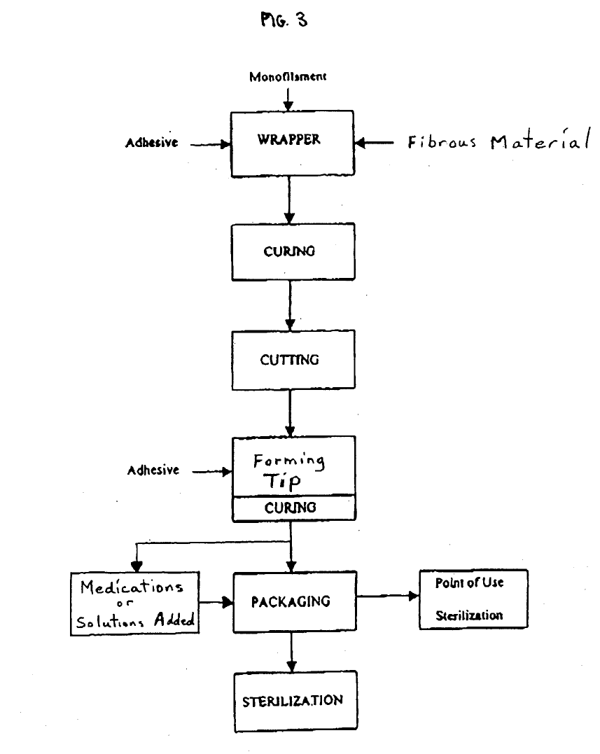

FIG. 3 shows a flow chart for the process for making the cleaning device.

### Preferred Embodiment of the Process for Making the Cleaning Device

1. The non-fibrous core is placed in the wrapping apparatus. The wrapping machine may then rotate the non-fibrous core after the core is lightly coated with adhesive. While the wrapping machine rotates the core, the fibrous material in thread form is spun around the core. Alternatively, the core may be advanced non-rotationally, and the fibrous material in thread from is helically rotated around the core to wrap it. In either embodiment, the wrapping machine tightly winds the fibrous thread around the core in a helical manner. In the preferred embodiment, the diameter of the non-fibrous core ranges from 0.010 to 0.060 inches and the diameter of the fibrous thread ranges from 0.001 to 0.010 inches. In the further preferred embodiment, the diameter of the non-fibrous core ranges from 0.015 to 0.025 inches and the diameter of the fibrous thread ranges from 0.003 to 0.007 inches. In the most preferred embodiment, the diameter of the non-fibrous core ranges from 0.018 to 0.020 inches and the diameter of the fibrous thread is 0.005 inches. Based on the ever increasing variety of piercings, cores of varying diameters may be used without deviating from the present invention.

The non-fibrous core may be made from a number of products, including but not limited to monofilaments of nylon, polypropylene, plastic, polyester, or natural non-fibrous material. In the preferred embodiment, the non-fibrous core is made of a nylon monofilament. The non-fibrous core is rigidly flexible in that it is rigid enough to be pushed into and through body piercings, while at the same time, flexible enough to easily move through curved areas. The core must be flexible enough so that it will not snap or break and create sharp edges. The preferred stiffness of the non-fibrous core in relation to the diameter range listed above ranges from 75 to 5,000 in/lb. The more preferred stiffness of the monofilament core ranges from 2,500 in/lb to 4,500 in/lb. The most preferred stiffness of the monofilament core ranges from 3,000 in/lb to 4,000 in/lb.

The fibrous thread wrapped around the non-fibrous core may also be made up of any number of materials, including, but not limited to natural fibers such as cotton, or synthetic fibers such as polyester. In the preferred embodiment the fibrous thread is made of unmercerized cotton. Unmercerized cotton is preferred because its textured surface creates sufficient friction for the cleaning device and improves its cleaning capabilities, while at the same time not abrading or causing discomfort to the user. In the context of the present invention, textured means having surface roughness, but not where the surface is so rough as to cause discomfort. Other textured fibrous threads may be used as listed above.

The adhesive used in the process may include any suitable adhesive. It is preferred that the adhesive be non-toxic and it is further preferred that the adhesive be USP Class VI certified. The most preferred adhesive to be applied to the monofilament is heat curable non-toxic epoxy. Alternatively, depending upon the adhesive chosen, the adhesive may be curable by ultraviolet or infrared radiation.

2. Once the fibrous thread has been wrapped around the non-fibrous core, the uncut length of wrapped core is heated to cure the adhesive. In a preferred embodiment, the wrapped core is passed through a furnace at 250° F. for three minutes to initiate curing of the epoxy. The time and temperature may vary inversely, however the temperature range is limited between the minimum heat necessary to cure the adhesive and the maximum heat that may soften the core and distort the winding. Alternatively, the adhesive may be cured by ultraviolet or infrared radiation, depending on the adhesive used.

3. After curing, the wrapped core is cut to a suitable length. In the preferred embodiment, the length may range from 2.0 to 7.0 inches with a most preferred range of 3.0 to 3.5 inches. Alternatively, the wrapped core may be cut after a short length of wrapping is stripped from the core. A stripped segment is formed on the wrapped core by stripping a short length of wrapping from the core at a specific length, corresponding to the desired length of the device. The preferred length of the stripped non-fibrous core may range from 0.01 to 0.20 inches, with a more preferred length of 0.01 to 0.10 inches and a most preferred length of 0.05 inches. The wrapped core is cut at the point where the stripped segment ends. If the wrapped core is cut before the wrapping is stripped, the same procedure is used, except the procedure targets one end of the cut wrapped core.

4. Once the wrapped core is cut, a rounded tip on the leading end may be formed. The stripped end of the wrapped core is dipped into a reservoir of uncured adhesive similar or equivalent to the one used to bind the fibrous thread to the core. Plastic or other related material is preferred because it can possess a smooth, non-porous surface that facilitates sliding into a body piercing to be cleaned. Additionally, a rounded tip formed of plastic or other related material is more economical then a metal tip and is not cold and uncomfortable when used by the consumer.

The adhesive should be warmed to a temperature where its viscosity is minimized so that a smooth surface rounded tip is formed on the end of the core. The preferred temperature for proper viscosity when using heat-cured epoxy is about 100° F. Proper viscosity is necessary to provide the proper texture for the rounded tip. If the temperature is too low, the rounded tip becomes too rough and may cause discomfort or irritation when being inserted into the body piercing.

By dipping the stripped core into the adhesive, the rounded tip is directly attached to the non-fibrous core as opposed to the outer fibrous material. Forming the tip directly on the core allows the rounded tip to have a smaller diameter. The small diameter is necessary for ease of insertion and comfort when inserting the device into a body piercing.

After the rounded tip is formed, the wrapped core is heated to cure the rounded tip. In a preferred embodiment, the wrapped core is passed through a furnace at 250° F. for 3 minutes to be cured. Once again the time and temperature may vary inversely, however the temperature range is limited between the minimum heat necessary to cure the adhesive and the maximum heat that may soften the core and distort the winding. Alternatively, the tip may be cured by ultraviolet or infrared radiation depending on the adhesive used.

5. Once the leading end with a round tip has been formed, the cleaning device may be prepared and packaged for sale. The preparation may include any number of steps. The cleaning device may be medicated to suit the particular needs of different consumers. Such medication may include, but is not limited to, water, saline, alcohol, peroxide, iodine, ascetic acid, benzalkonium chloride; sodium chloride, various anesthetics and/or other cleansing or sanitizing compositions. The medication may be added to the cleaning device before packaging or may be included with the cleaning device inside the packaging. Generally, the cleaning devices may be packaged individually or as multiple devices in a package or a kit. Once the cleaning devices are placed in their packages, the packages are heat sealed and sterilized. Alternatively, the sealed package may include an indicator signifying sterilization and be sterilized by the consumer at the point of use, such as the home, piercing studios, hospitals and doctor's offices.

### Preferred Embodiment of a Method for Using the Cleaning Device

The cleaning device may be used with the human body specifically, but is not limited to that use. More specifically, the cleaning device may be used to clean body piercings; inserted into vacant piercings as a place holder during MRI, CT scans, or ultrasound examinations or the like, as well as surgical procedures, enabling patients to reinsert jewelry easily after the procedure. The device may also be used to clean dental work such as bridge work; used to apply solutions and medications directly to the teeth and gums; modified with measuring markings and used to measure the depth of bed sores, surgical procedures and the like; and may be used as a culture swab for patients, lab's and doctors, among other uses. Veterinary uses are also envisioned and are similar to the uses in human applications. Additionally, the cleaning device may be used in environments outside the scope of the animal body, for example to clean intricate mechanical parts. The most preferred use is a cleaning device for body piercings of humans.

The device may be used with any body piercing. To use the cleaning device, remove the device from its sterilized packaging. Insert the rounded tip of the leading end into the body piercing to be cleaned. Push the device through the body piercing with a slight gentle rotating, twisting or spinning motion. Once the leading end emerges from the body piercing, pull the device from the leading end through the body piercing with a slight rotating, twisting or spinning motion, in only one direction. Do not move the cleaning device back and forth. Using the device in only one direction reduces the risk of irritation or infection and removes any particles that have adhered to the swab providing more for effective cleaning. If the piercing has a significantly greater diameter than the cleaning device, rotate the cleaning device once around the inside circumference of the piercing until the entire inside has been cleaned, going around one full circle, to achieve full cleansing of the body piercing. Once the cleaning device is pulled all the way through the body piercing, dispose of the cleaning device.

The cleaning device is easy to use, yet is useful to both body piercing professionals and home users alike. The rounded tip allows for smooth and easy insertion into the body piercing, without irritation and discomfort to the user. Additionally, the one-time use and one-way direction aspects of the cleaning device increase the cleaning utility of the device and decrease the possibility of infection or aggravation of the body piercing, by not reinserting fluids and particles that have adhered to the device during its one way pass through the body piercing.

Although the present invention has been described in detail with particular reference to preferred embodiments thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only, and do not in any way limit the invention, which is defined only by the claims.

What is claimed is:

1. A cleaning device for body piercings, comprising:
a disposable non-fibrous core wrapped with a textured fibrous material having a predetermined diameter;
a leading end for threading through a body piercing;
a following end;
said leading end having a tip with a diameter greater than said core;
said following end having a diameter equal or less than said core.

2. The device of claim 1, wherein said core is a monofilament.

3. The device of claim 1, wherein said core is rigidly flexible.

4. The device of claim 1, wherein said core has a stiffness range of 2,500 in/lb to 4,500 in/lb.

5. The device of claim 1, wherein said fibrous material is synthetic, naturally derived and combinations thereof.

6. The device of claim 1, wherein said tip is integral with said core.

7. The device of claim 1, wherein said tip is attached to said core.

8. The device of claim 1, wherein said tip is bonded to said core.

9. The device of claim 1, wherein the device is medicated.

10. A method of using a device for cleaning body piercings, where the device of claim 1 is inserted, leading end first, into the body piercing to be cleaned, then pulled through the body piercing in only one direction until the following end exits the body piercing.

11. A method for making a cleaning device for body piercings, comprising:
wrapping a disposable non-fibrous core with a textured fibrous material to form a wrapped core with a predetermined diameter, where the device has leading end for threading through a body piercing and a following end;
forming said leading end with a tip having a diameter greater than said core and said following end having a diameter equal to or less than said core.

12. The method of claim 11 where the stiffness of the non-fibrous core is 2,500 in/lb to 4,500 in/lb.

13. The method of claim 12 where the non-fibrous core is coated lightly in adhesive and the fibrous material is wound tightly around the core in a helical manner so that the adhesive binds the fibrous material to the non-fibrous core.

14. The method of claim 13 where the core wrapped with fibrous material is cured.

15. The method of claim 14 where the wrapped core is cut to a length of 2.0 to 7.0 inches.

16. The method of claim 15 where a rounded tip is formed on the leading end of the cleaning device.

17. The method of claim 16 where the rounded tip is formed by stripping a short length of fibrous wrapping from the leading end of the wrapped non-fibrous core and dipping the stripped end into a reservoir of warmed uncured adhesive, then removing the dipped end from the reservoir and curing the adhesive.

18. The method of claim 17 where the cleaning device is packaged in a heat sealed package and sterilized.

19. The method of claim 20 where the cleaning device is medicated.

20. A cleaning device for body piercings, comprising:
a non-fibrous core;
a textured fibrous material wrapped around said core forming a wrapped core with a predetermined diameter;
a leading end formed with a tip that has a diameter greater than said core;
a following end formed with a diameter equal or less than said core.

21. The device of claim 20 where the non-fibrous core is coated lightly in adhesive and the fibrous material is wound tightly around the core in a helical manner so that the adhesive binds the fibrous material to the non-fibrous core.

22. The device of claim 21 where the core wrapped with fibrous material is cured.

23. The device of claim 22 where the wrapped core is cut to a length of 2.0 to 7.0 inches

24. The device of claim 23 where a rounded tip is formed on the leading end of the cleaning device.

25. The device of claim 24 where the rounded tip is formed by stripping a short length of fibrous wrapping from the leading end of the wrapped non-fibrous core and dipping the stripped end into a reservoir of warm uncured adhesive, then removing the dipped end and curing the adhesive.

26. The device of claim 25 where the device is packaged in a heat-sealed package and sterilized.

27. The device of claim 26 where the device is medicated.
